(19) United States
(12) Patent Application Publication
(10) Pub. No.: US 2024/0197506 A1
(43) Pub. Date: Jun. 20, 2024

(54) AN IMPLANTABLE DEVICE FOR BRANCHED LUMENS WITH ASSOCIATED SYSTEMS AND METHODS

(71) Applicant: W. L. Gore & Associates, Inc., Newark, DE (US)

(72) Inventors: Dustin C. Burkart, Bellemont, AZ (US); Martin J. Sector, Phoenix, AZ (US); John Voorhees, Flagstaff, AZ (US)

(21) Appl. No.: 18/287,151

(22) PCT Filed: Feb. 22, 2022
(86) PCT No.: PCT/US2022/017251

§ 371 Date: Oct. 16, 2023

Related U.S. Application Data
(60) Provisional application No. 63/176,031, filed on Apr. 16, 2021.

(51) Int. Cl. A61F 2/954; A61F 2/00; A61F 2/06; A61F 2/07; A61F 2/966

(52) U.S. Cl. CPC A61F 2/954; A61F 2/07; A61F 2002/0081; A61F 2002/061; A61F 2002/067; A61F 2002/9665

(57) ABSTRACT

An implantable device comprising including a tubular member and a stent member supporting the tubular member. The tubular member includes a first end and a second end, the tubular member forming a primary lumen having a first opening at the first end of the tubular member and a second opening at the second end of the tubular member. The tubular member includes a column positioned within the primary lumen and forming a secondary lumen, the tubular member defining a plurality of apertures opening into the secondary lumen at positions longitudinally between the first end and the second end of the tubular member, the column having a column opening.

### CROSS-REFERENCE TO RELATED APPLICATION

This application is a national phase application of PCT Application No. PCT/US2022/017251, internationally filed on Feb. 22, 2022, which claims the benefit of Provisional Application No. 63/176,031, filed Apr. 16, 2021, which are incorporated herein by reference in their entireties for all purposes.

### BACKGROUND

In numerous locations of the human anatomy, a primary bodily conduit (e.g., primary blood vessel) is connected with one or more secondary bodily conduits (e.g., branch or peripheral vessels) that branch off from the primary bodily conduit. In some cases the secondary bodily conduits convey fluid into the primary bodily conduit (e.g., venous conduits), while in other cases the secondary bodily conduits convey fluid away from the primary bodily conduit (e.g., arterial conduits).

The human vasculature includes many examples of primary bodily conduits that have secondary branches. One example of a primary bodily conduit is the aorta. In the abdominal aorta, a plurality of arteries branch off from the aorta.

Bodily conduits and/or circulatory (or other bodily) systems with which they are associated may suffer from various diseases, defects and conditions, or may otherwise benefit from augmentation using one or more implantable prosthetic conduits, including grafts, stent-grafts, filters, anastomosis devices, prosthetic valves, and others. Generally, such implantable conduits have tubular forms configured to convey fluid in the body.

### SUMMARY

Disclosed herein are examples of an implantable device.

According to one example, (“Example 1”), an implantable device includes a tubular member having a first end and a second end, the tubular member forming a primary lumen having a first opening at the first end of the tubular member and a second opening at the second end of the tubular member, the tubular member includes a column positioned within the primary lumen and forming a secondary lumen, the tubular member defining a plurality of apertures opening into the secondary lumen at positions longitudinally between the first end and the second end of the tubular member, the column having a column opening; and a stent member supporting the tubular member.

According to another example (“Example 2”), further to Example 1, the tubular member includes a first graft member defining the primary lumen and a second graft member coupled to the first graft member forming the column and defining the secondary lumen between the first and second graft members.

According to another example (“Example 3”), further to Example 2, the secondary lumen opens into the primary lumen at the proximal opening of the secondary lumen.

According to another example (“Example 4”), further to any of the preceding Examples, the secondary lumen is collapsible.

According to another example (“Example 5”), further to any of the preceding Examples, the secondary lumen is unsupported by a stent.

According to another example (“Example 6”), further any of the preceding Examples, the stent member includes a plurality of stent rings spaced longitudinally along the tubular member.

According to another example (“Example 7”), further to Example 6, each of the plurality of apertures is separated from each other by at least one of the plurality of stent rings.

According to another example (“Example 8”), further to any of the preceding Examples, each aperture of the plurality of apertures includes a circular shape or a shape defining a rounded portion and a substantially flat portion.

According to another example (“Example 9”), further to any of the preceding Examples, the tubular member includes a plurality of secondary lumens extending longitudinally along at least a portion of the tubular member and circumferentially spaced from each other.

According to another example (“Example 10”), further to any of the preceding Examples, the implantable device further includes a constraining member receiver positioned surrounding at least a portion of the stent member.

According to another example (“Example 11”), further to any of the preceding Examples, the tubular member includes a scallop at the first end.

According to another example (“Example 12”), further to any of the preceding Examples, the secondary lumen extends along a secondary lumen axis that extends longitudinally at an angle greater than zero relative to an axis of the primary lumen.

According to another example (“Example 13”), an implantable device includes a tubular member having a first end and a second end, the tubular member forming a primary lumen having a first opening at the first end of the tubular member and a second opening at the second end of the tubular member, the tubular member including a plurality of columns each defining a secondary lumen, each column of the plurality of columns being circumferentially spaced from each other, each secondary lumen having an aperture defined through the tubular member at a position between the first end and the second end and having a column opening proximate the second end of the tubular member, each column being operable to collapse under hydrostatic pressure; and a stent member supporting the tubular member.

According to another example (“Example 14”), a delivery system includes an implantable device including a main body and a side branch, the main body including a tubular member and a stent member supporting the tubular member, the tubular member forming a primary lumen having a first opening and a second opening, the tubular member forming a secondary lumen having an aperture defined through the tubular member and having a proximal opening proximal the second opening of the tubular member; an elongate member having a first end and a second end, the main body of the implantable device being positioned at the first end of the elongate member, the elongate member defining a delivery lumen extending along at least a portion of a longitudinal length of the elongate member and through which the side branch is operable to be delivered; a catheter olive positioned at the first end of the elongate member such that the main body of the implantable device is positioned longitudinally between a leading tip of the catheter olive and the second end of the elongate member; a constraining member positioned about the main body and constraining the main body to a delivery configuration; and a secondary constraining member positioned about the stent of the main body, the secondary constraining member operable to constrain the main body to a partially constrained configuration.

According to another example (“Example 15”), further to Example 14, the delivery system further includes a guide member extending from the delivery lumen of the elongate member, through the secondary lumen of the tubular member from the proximal opening and through the aperture, and coupled to the catheter olive.

According to another example (“Example 16”), further to Example 15, the delivery system further includes an exchange catheter coupled to the guide member and operable to be advance along the guide member.

According to another example (“Example 17”), further to Example 16, the delivery system further includes a first guidewire operable to be delivered through the exchange catheter; a curved catheter operable to be advanced over the first guidewire; and a second guidewire that is stiffer than the first guidewire, the second guidewire operable to be advanced through the curved catheter, the side branch operable to be advanced to a target site along the second guidewire.

According to another example (“Example 18”), a delivery system includes an implantable device including a main body and a side branch, the main body including tubular member and stent member supporting the tubular member, the tubular member forming a primary lumen having a first opening and a second opening, the tubular member including a column defining a secondary lumen and having an aperture defined through the tubular member and having a column opening proximal the second opening of the tubular member; an elongate member having a first end and a second end, the main body of the implantable device being positioned proximal to the first end of the elongate member, the elongate member defining a lumen through which the side branch is operable to be delivered; a catheter olive positioned at the first end of the elongate member such that the main body of the implantable device is positioned longitudinally between a leading tip of the catheter olive and the second end of the elongate member; a constraining member positioned about the main body and constraining the main body to a delivery configuration; and a guide member extending from the lumen of the elongate member, through the secondary lumen of the tubular member from the proximal opening and through the aperture, and coupled to the catheter olive.

According to another example (“Example 19”), the delivery system further includes a secondary constraining member positioned about the stent of the main body, the secondary constraining member operable to constrain the main body to a partially constrained configuration.

According to another example (“Example 20”), further to Example 19, the stent member of the main body includes a plurality of stent rings, wherein the secondary constraining member includes a plurality of wires, wherein a wire of the plurality of wires corresponds to a corresponding stent ring of the plurality of stent rings.

According to another example (“Example 21”), a method of delivering an implantable device to a target site including a main vessel and side branch vessels, the method including advancing a main body of the implantable device to the main vessel of the target site, the main body being constrained by a constraining member about an elongate member proximate a first end of the elongate member, the main body including a tubular member having a first end and a second end, the tubular member forming a primary lumen having a first opening at the first end of the tubular member and a second opening at the second end of the tubular member, the tubular member having a plurality of columns each defining a secondary lumens extending along at least a portion of a longitudinal length of the tubular member and each having a plurality of apertures defined through the tubular member at positions between the first end and the second end of the tubular member, each secondary lumen having a column opening proximal the second end of the tubular member, the tubular member including a scallop at the first end of the tubular member, the main body including a stent member supporting the tubular member and operable to be configured in a delivery configuration and in a deployed configuration; positioning the scallop of the tubular member proximate a side branch vessel; releasing the main body from the constraining member such that the main body is operable to be expanded from a delivery configuration to an expanded configuration; advancing an exchange catheter toward a side branch vessel, the exchange catheter advanced along a guide member extending from the elongate member, through one of the plurality of secondary lumens of the tubular member from the proximal opening and through a first end aperture of the plurality apertures, and releasably coupled to a catheter olive at the first end of the elongate member; introducing a first guidewire through the exchange catheter such that first guidewire extends beyond the first end aperture of the secondary lumen; retracting the exchange catheter away from around the first guidewire; selecting a target aperture of the plurality of apertures through which the side branch is delivered, including retracting the first guidewire is positioned at or through the target aperture; exchanging the first guidewire with a second, stiffer guidewire; advancing the side branch to a side branch vessel via the second guidewire; and deploying the side branch at the side branch vessel.

The foregoing examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 illustrates a side view of an implantable device having a main body and a plurality of side branches extending therethrough, according to some embodiments.

FIG. 2A Illustrates a perspective view of a main body of an implantable device including portals and a plurality of apertures, according to some embodiments.

FIG. 2B illustrates an end view of the main body of FIG. 2A demonstrating an opening of the portal positioned at the second end of the main body, according to some embodiments.

FIG. 2C illustrates a perspective view of a main body having apertures with a curved portion and a substantially straight portion, according to some embodiments.

FIG. 3 illustrates a side view of a delivery system for an implantable device including an elongate member, a catheter olive, guide members, constraining members, and a delivery handle, the implantable device positioned about the elongate member, according to some embodiments.

FIGS. 4A and 4B illustrate perspective views of an implantable device with constraining members operable to controllably constrain the implantable device, according to some embodiments.

FIGS. 5A-5C illustrate side view of guide members extending to a catheter olive, according to some embodiments.

FIG. 6 illustrates a perspective view of an example guide member extending through an implantable device, according to some embodiments.

FIG. 7 illustrates a cross section of an exchange catheter, according to some embodiments.

FIG. 8 illustrates a side view of the exchange catheter of FIG. 7 extending from an elongate member in a portal of an implantable device, according to some embodiments.

FIG. 9 illustrates a side view of a delivery handle for an implantable device, according to some embodiments.

FIG. 10 illustrates an end view of an elongate member including a plurality of delivery lumens, according to some embodiments.

FIGS. 11A-11C illustrate side views of an implantable device having a main body positioned in the abdominal aortic artery, a plurality of side branches positioned in renal arteries, and a plurality of limbs positioned in iliac arteries, according to some embodiments.

FIG. 12 is a perspective view of an implantable device showing side branches extending through a portion of the main body, according to some embodiments.

FIGS. 13-27 illustrate a delivery method for delivering an implantable device to a target vessel and side branches to side branch vessels, according to some embodiments.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. For example, such deviations may be attributable to measurement error or minor adjustments made to optimize performance. In the event it is determined that individuals having ordinary skill in the relevant arts would not readily ascertain values for such reasonably small differences, the terms “about” and “approximately” and similar terminology can be understood to mean plus or minus 10% of the stated value.

### DESCRIPTION OF VARIOUS EMBODIMENTS

Various aspects of the present disclosure are directed to apparatuses, systems, and methods that include an implantable device. The implantable device may be arranged within a patient for supporting one or more fluid passageway(s). The implantable device may be positioned in a main portion of fluid passageway (e.g., artery, vein, CSF passageway, and so forth) and include side branches that extend into side branch passageways within the patient. In certain instances, the implantable device may be a graft or stent graft that is arranged within the vasculature. The apparatuses, systems, and methods may be used in improving or assisting circulatory function and specifically that of side branches (e.g., leading to critical organs such as the kidneys). In one example, a patient may have an abdominal aortic aneurysm (“AAA”). The AAA may develop near the renal arteries and/or extend into the renal arteries. An implantable device may be used to help prevent enlargement and rupturing of the aneurysm while maintaining perfusion of the main artery and one or more side branch arteries, such as the renal arteries.

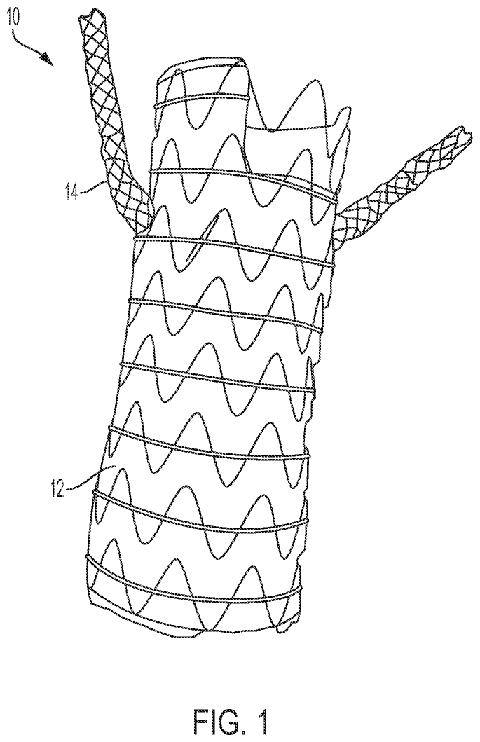

FIG. 1 illustrates a side view of an example of an implantable device 10 having a main body 12 and a plurality of side branches 14 extending therethrough. The implantable device 10 also includes the side branches 14 extending from the main body 12. The side branches 14 are separate from the main body (i.e., they are not integral with the main body 12). Because the side branches 14 are separate structures from the main body 12, the side branches 14 are coupled to the main body 12 to form the implantable device. For example, the main body 12 may be deployed in the abdominal aorta and the side branches 14 may be deployed in the renal arteries and extend into the main body 12 positioned in the abdominal aorta.

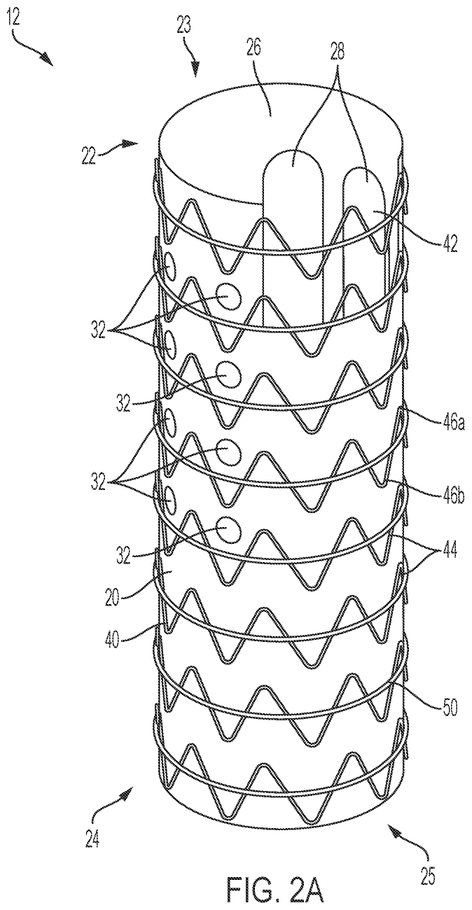

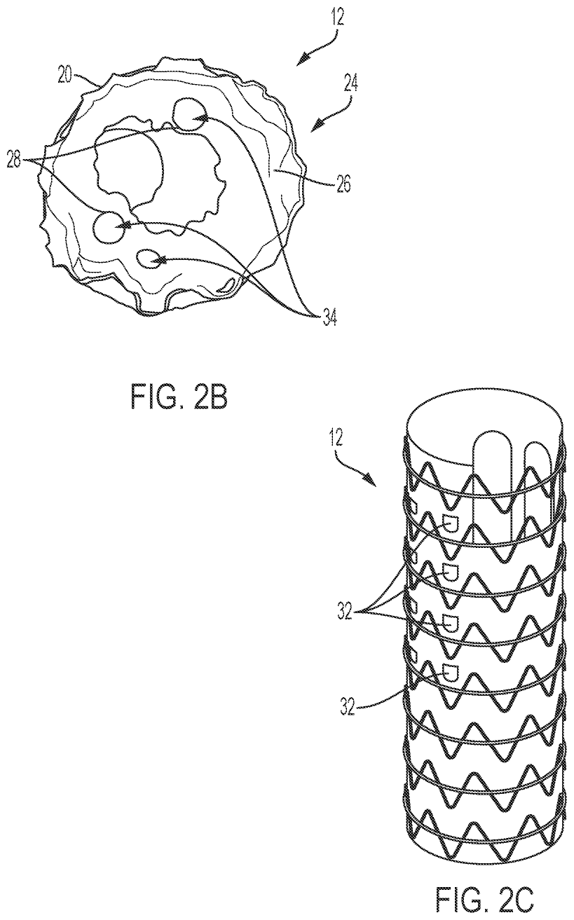

As shown in FIG. 2A, in some embodiments the main body 12 of the implantable device 10 includes a tubular member 20 and a stent member 40. As shown, the tubular member 20 has a first end 22 and a second end 24. The tubular member 20 forms a primary lumen 26 having a first opening 23 at the first end 22 and a second opening 25 at the second end 24 of the tubular member 20. The tubular member 20 includes a column 28 positioned within the primary lumen 26 and forms a secondary lumen 30 (see FIG. 2B). The tubular member 20 defines an aperture 32 into the secondary lumen 30 at a position longitudinally between the first end 22 and second end 24 of the tubular member 20. The column 28 defines a column opening 34 (see FIG. 2B) proximal the second end 24 of the tubular member 20. The stent member 40 supports the tubular member 20 in such a manner that the implantable device is operable to be configured in a delivery configuration and in a deployed configuration, or to be transitioned from a delivery configuration toward a deployed configuration.

In some embodiments, the tubular member 20 includes a first graft member 41 defining the primary lumen 26 and a second graft member 42 coupled to the first graft member 41 to form the column 28 defining the secondary lumen 30 between the first graft member 41 and the second graft member 42. For example, the first graft member 41 includes graft material formed in the shape of a tube to define the primary lumen 26. And, the second graft member 42 optionally includes graft material that is coupled to the first graft member 41 (e.g., via boding, adhesive, or by otherwise being coupled together) to form the secondary lumen 30. The graft materials of the first and second graft members 41, 42 may be the same material or different materials as desired. Though some materials may provide certain advantages over others, a variety of suitable graft materials may be implemented, and generally any suitable graft material may be implemented including those materials discussed herein.

In some embodiments the secondary lumen 30 extends at least partially along a longitudinal length of the main body 12. The secondary lumen 30 of the column 28 opens into the primary lumen 26 at the proximal opening of the secondary lumen 30. In some embodiments, the column 28 extends to the second end 24 of the tubular member 20 such that the column opening 34 is positioned at or coplanar with the second opening 25 of the tubular member 20. In other embodiments, the column 28 extends toward the second end 24 of the tubular member 20 such that the column opening 34 is longitudinally spaced from the second opening 25 of the tubular member 20. In embodiments including a plurality of columns 28, the column openings 34 may be positioned at the same longitudinal length across, or in different terms, at the same longitudinal position along, the tubular member 20 or they may be staggered at two or more longitudinally-spaced positions along the length of the tubular member 20.

In some embodiments, the column 28, and consequently the secondary lumen 30 are collapsible. For example, the column 28 may be unsupported by a stent member, although supported, collapsible embodiments are also contemplated. Lack of a support, or a suitably configured support, may allow the column 28 to be collapsed (radially collapsed) to seal the aperture 32 and limit the leaking or other passing of fluids (e.g., blood) through the aperture 32. In some embodiments, the pressure (e.g., hydrostatic pressure, fluid pressure gradients, and/or pressure exerted by fluids in motion) that is exerted by the fluid collapses the column 28 such that the column coats or seals against the tubular member 20 to limit flow through the secondary lumen 30 and consequently the aperture 32.

As illustrated in FIG. 2A, the column 28 may be sealed or closed near the first end 22 of the tubular member 20, or, in some embodiments not shown, at the first end 22. The secondary lumen 30 thus is operable to provide fluid communication between the exterior surface of the tubular member 20 between the first and second end 22, 24 and the primary lumen 26, for example, when the column 28 is patent. In some embodiments, the tubular member 20 may include a column 28 that is unsealed (i.e., includes an opening) near the first end of the tubular member 20. In such embodiments, an elongate member such as a delivery catheter may be positioned through the column 28. Referring to FIG. 2B, an end view of the main body 12 is shown with the column opening 34 positioned proximate the second end 24 of the main body 12. In some embodiments, columns 28 extend to the second end 24 of the main body 12. As illustrated, the secondary lumen 30 may be contained within the primary lumen 26.

Referring again to FIG. 2A, the main body 12 includes the stent member 40. The stent member 40 may be formed of any suitable material as is discussed hereafter. The stent member 40 is operable to support the tubular member 20. The stent member 40 may be compressed into a delivery configuration and may be expanded into an expanded configuration, such as at deployment. The stent member 40 may be a self-expanding stent or a balloon expandable stent. As illustrated, the stent member 40 includes a plurality of stent rings 44. Each stent ring 44 circumferentially supports the tubular member 20 at a longitudinal position along the length of the tubular member 20. For example, each stent ring 44 is longitudinally spaced from an adjacent stent ring 44. The stent rings 44 may each include apices 46 with first apices 46a pointing toward the first end 22 and second apices 46b pointing toward the second end 24. Various other configurations of stent members 40 are contemplated herein including, but not limited to, helical stents (including undulating helical stents, diamond pattern stents, and others).

As illustrated in FIG. 2A, the tubular member 20 includes a plurality of apertures 32 spaced along the longitudinal length of the main body 12. The apertures 32 may be positioned such that at least one stent ring is between the two longitudinally adjacent apertures 32. For example, a column 28 may include apertures 32 through the tubular member 20 such that the apertures 32 are longitudinally spaced along the main body 12. The apertures are all in fluid communication with the secondary lumen 30 of the column 28. The apertures 32 provide access points for the secondary branch at various longitudinal lengths along the main body 12.

Referring to FIG. 2C, the apertures 32 may be formed in a variety of shapes and size including circular profiles, a profile with a rounded edge and a substantially flat edge, ovular profiles, and so forth. The various shapes and sizes may be implemented to accommodate various side branches 14 and configurations such as angle of exit of the side branches 14 from the main body 12 at the apertures 32. In some embodiments not shown, the apertures 32 may be irregularly spaced along the longitudinal length of the column 28. Furthermore, in some embodiments not shown, the apertures 32 may be circumferentially spaced within a column 28. For example, the apertures 32 may be staggered circumferentially and/or longitudinally.

In some embodiments, the main body 12 may include a plurality of columns 28. For instance, the main body 12 may include two columns 28 that are circumferentially spaced from each other in order to deploy two side branches 14 into the side branch lumens of the patient's anatomy. Furthermore, the main body 12 may include a plurality of columns 28 that are associated with each side branch lumen of the patient's anatomy. For example, if the main body 12 is to be deployed in the abdominal aorta and the side branches 14 are to be deployed into the renal arteries, each patient may have a various positions circumferentially at which the renal arteries enter the aorta.

By having a plurality of columns 28 through which each side branch 14 may be deployed, the surgeon may select the appropriate columns 28 that best conform to the patient's native anatomy without applying torsion to the vessels when the implantable device 10 is deployed. Thus, in one example, the main body 12 may include three columns 28 on one circumferential side of the tubular member 20 and three more columns 28 on an opposite circumferential side of the tubular member 20. Each column 28 is circumferentially spaced from the adjacent column 28 about the circumference of the tubular member 20. It is contemplated that any number of columns 28 and the spacing of the columns 28 may be implemented, including one, two, three, four, five, six, seven, eight, or more columns 28 which may be spaced equally or variably about the circumference of the tubular member 20. It is further contemplated that the specific spacing may be determined by surveying the average circumferential spacing of side branches for a particular implementation in a sample population of patients to determine the spacing of the columns 28. Circumferential spacing of the column 28 allows for clocking of the main body 12 within the patient's anatomy with increased positions for appropriately positioning the side branches 14 into the side branch vessels. As used herein, the term “clocking” refers to the ability to position features at a desired location about a circumference of an object. This ability to clock the one or more columns 28 can be further advantageous for use with visualization, for example when the procedure is being performed via fluoroscopy. This simplifies placement by providing several entry points when dealing with the two-dimensional planes shown by visualization techniques and for parallax associated with such visualization. In some embodiments, the columns 28 may be irregularly spaced about the circumference of the main body 12 (e.g., non-uniform spacing between the columns 28). In some embodiments not shown, the column 28 extends longitudinally and at angle greater than zero relative to the main body 12 longitudinal axis. For example, the secondary lumen 30 extends along a secondary lumen axis that extends longitudinally at an angle greater than zero relative to an axis of the primary lumen 26 (e.g., helically about the main body 12).

Referring again to FIG. 2A, the main body 12 may include a constraining member receiver 50 positioned surrounding at least a portion of the stent member 40. For example, in those embodiments including a plurality of stent rings 44, a corresponding constraining member receiver 50 is positioned about each stent ring 44. The constraining member receiver 50 may be formed from a variety of materials including graft materials, fibers, and so forth. The constraining member receiver 50 is operable to receive constraining members that can be retracted to partially constrain or collapse the stent rings 44 as is discussed hereafter.

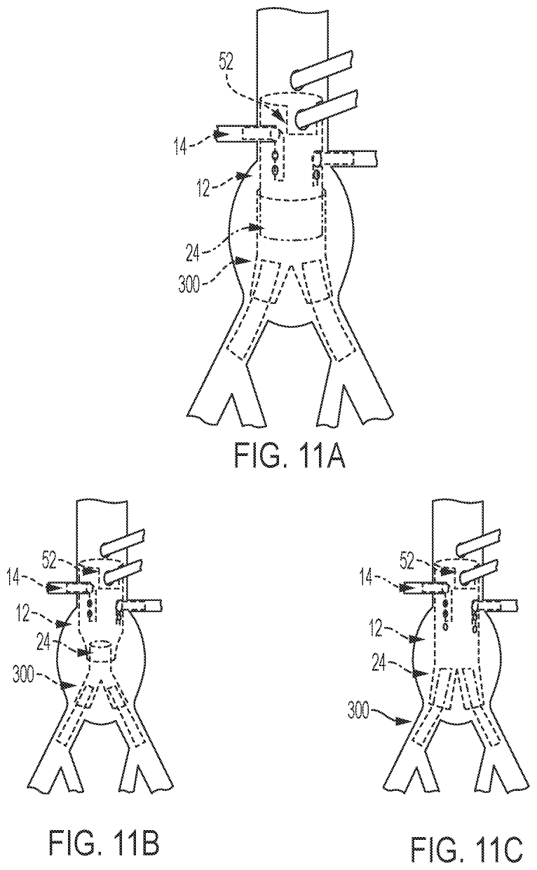

In some embodiments, the tubular member 20 may include a scallop 52 at the first end 22. The scallop 52 is a facilitates placement of the tubular member 20 in a lumen including a side branch lumen that does not need a prosthetic side branch deployed. For example, when the implantable device 10 is positioned in the abdominal aorta and the superior mesenteric artery does not need a side branch 14 deployed therein, the scallop 52 may be positioned over the entrance into the superior mesenteric artery without blocking or restricting blood perfusion therethrough (see FIGS. 11A-11C). The scallop 52 may include various shapes including straight edge profiles, curved profiles, and combinations thereof.

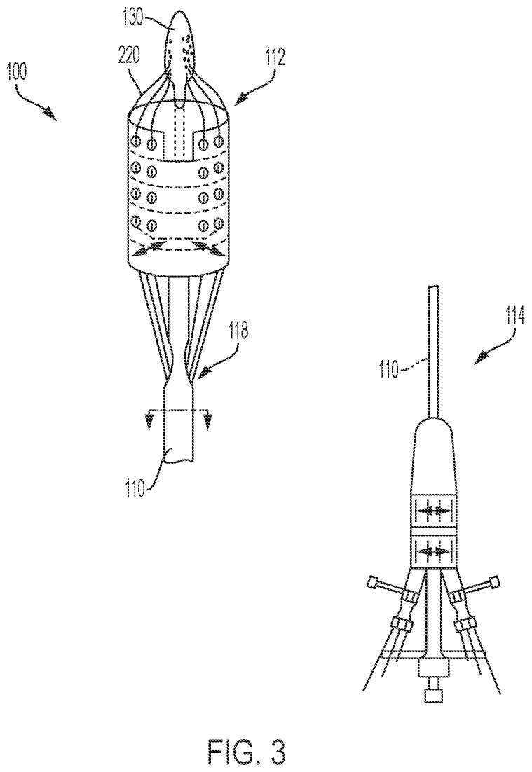

Referring now to FIG. 3, a delivery system 100 is provided for delivering and deploying the implantable device 10. Although the delivery system 100 and implantable device 10 may be discussed with reference to specific implantation sites such as the abdominal aorta, it is understood that the delivery system 100 and implantable device 10 may be implemented at various sites and in various systems. Furthermore, it is understood that the delivery system 100 may also be implemented with other implantable devices not described herein and therefore is not limited for use with the specific embodiments disclosed and otherwise contemplated herein. However, for the purposes of illustration, the delivery system 100 is discussed with respect to the implantable device 10 discussed herein.

The delivery system 100 is operable to facilitate placement of the implantable device 10 at a branched lumen, allow for at least partial collapse and re-expansion of the main body 12 of the implantable device 10 once deployed, and fenestration of the main body 12 with a side branch 14 for implantation of the side branch 14 at the side branch lumen.

The delivery system 100 is provided with an elongate member 110 (e.g., a delivery catheter), a catheter olive 130, a constraining member 150, a secondary constraining member 170, and a guide member 190. The delivery system may also include a delivery handle 200 for operating, controlling, and otherwise manipulating the delivery system 100 and its various components.

The elongate member includes a first end 112 and a second end 114, the main body 12 of the implantable device 10 being positioned at the first end 112 of the elongate member 110. Referring to FIG. 10, the elongate member 110 defines a delivery lumen 116 extending along at least a portion of a longitudinal length of the elongate member 110 and through which the side branch 14 is operable to be delivered. The delivery lumen 116 is sized to appropriately accommodated the side branch 14 and the delivery components. For example, the delivery lumen 116 may by sized at about 8 Fr. In other embodiments, the delivery lumen is from about 4Fr to about 12 Fr. In those embodiments in which the implantable device 10 includes two side branches 14, the elongate member may include two delivery lumens 116 for the respective side branches 14. Any number of delivery lumens 116 may be implemented for the respective number of side branches 14 to be deployed. As illustrated in FIG. 3, the elongate member 110 may include openings 118 spaced from the first end 112 of the elongate member. The openings 118 may be positioned either in the primary lumen 26 of the main body 12 when the main body is positioned about the elongate member 110 or spaced from second end 24 of the main body 12 (as illustrated in FIG. 3). This allows access to the column 28 and/or apertures 32 for placing and implanting the side branches 14 with the main body 12. The elongate member 110 may further include a constraining member lumen 120. The constraining member lumen 120 is operable to allow control of the constraining member 150 for releasing the main body 12 from the constrained configuration. The elongate member may further include a lock wire lumen 122 and secondary constraining member lumens 124. A lock wire and constraining wires may extend through the respective lumens 122, 124. In some embodiments not shown, the side branches 14 may be delivered in independent catheters (not shown).

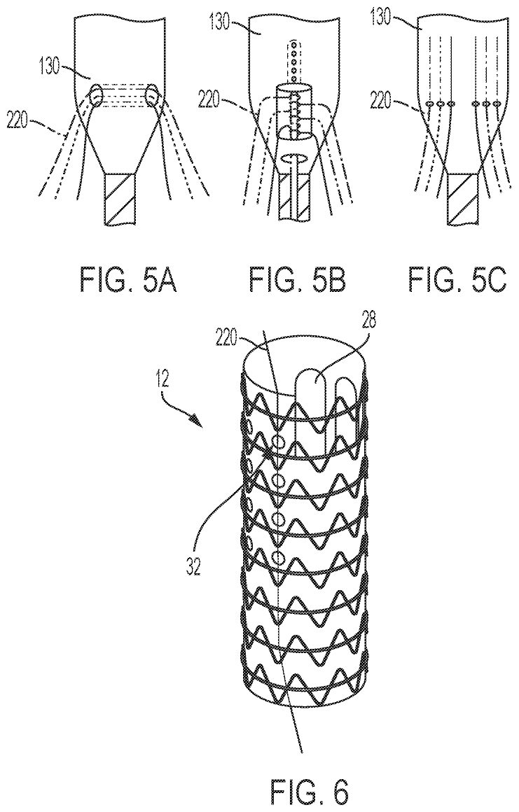

A catheter olive 130 positioned at the first end 112 of the elongate member 110 such that the main body 12 of the implantable device 10 is positioned longitudinally between the catheter olive 130 and the second end 114 of the elongate member 110. Although an embodiment of the catheter olive 130 is depicted in the drawings, it is in the scope of the disclosure that any catheter olive 130 may be implemented within the scope of this disclosure. The catheter olive 130 may be implement to atraumatically advance the delivery system 100 through the patient and to dilate the surrounding anatomy where appropriate. For example, the catheter olive may include a leading tip which is advanced first through the patient's anatomy. Referring to FIGS. 5A-5C, the catheter olive 130 may include a guide member retainer 132. The guide members 220 will be discussed in more detail hereafter. However, the guide member retainer 132 may comprise a passage through which the guide members 220 pass (see FIG. 5A). In this embodiment, the guide members 220 may pass through the catheter olive 130 and extend back through an aperture 32 of another, oppositely positioned column 28. The guide member retainer 132 may be operable to releasably retain a lock wire 230 to which the guide members 220 may be coupled (see FIG. 5B). The lock wire 230 may be controlled via the lock wire lumen 122. The guide member retainer may be operable to received and releasably retain ends of the guide members 220, for example via a friction fit or other coupling (see FIG. 5C). Various embodiments of catheter olives 130 may be implemented specifically for coupling the guide members 220 (e.g., the guide member retainers 132). Such embodiments include those discussed in U.S. Pat. Pub. No. 2020/0046534 by Chung et al., filed Aug. 13, 2019, the content of which is hereby expressly incorporated by reference.

A constraining member 150 is positioned about the main body 12 and constraining the main body to a delivery configuration. Any number of constraining members 150 may be implemented, and may include a knit, tubular, or any other releasable structure that can be selectively released or actuated to allow expansion of the device. Such structures include those discussed in U.S. Pat. No. 6,224,627 to Armstrong granted Jun. 15, 1998 and U.S. Pat. No. 7,753,945 to Bruun granted Jul. 13, 2010, the content of which is herein expressly incorporated by reference in their entireties. The constraining member 150 is removed and the main body 12 is operable to expand (e.g., self-expanding) or be expanded (e.g., balloon expandable) to an expanded configuration. The constraining member 150 may be controlled (e.g., released) from the main body 12 via a deployment line (not shown) extending through the constraining member lumen 120.

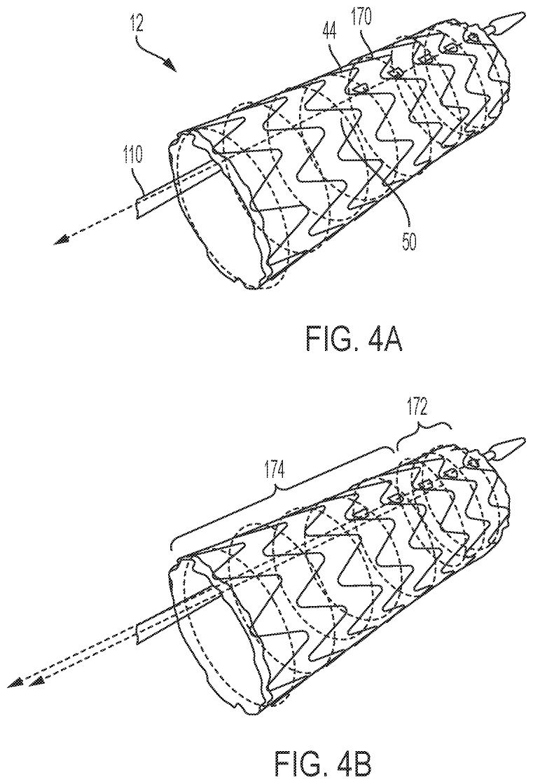

A secondary constraining member 170 positioned about the stent member 40 of the main body 12. In those embodiments including a plurality of stent rings 44, the delivery system 100 may include a plurality of secondary constraining members 170 corresponding to each stent ring 44. Referring to FIGS. 4A and 4B, the secondary constraining member 170 is operable to constrain the main body 12 to a partially constrained configuration. For example, after the constraining member 150 has been removed from the main body 12 and the main body has been expanded to an expanded configuration, the secondary constraining member 170 can be tensioned to at least partially collapse the main body to a partially constrained configuration. The secondary constraining members 170 are positioned within the constraining member receiver 50 of the main body 12. Various embodiments of constraining member receivers 50 may be implemented, including those discussed in U.S. Pat. Pub. No. 2018/0036011 by Lehnhardt et al. filed Aug. 7, 2017, the contents of which are herein expressly incorporated by reference in their entirety. The constraining member receivers 50 position the secondary constraining members 170 about the corresponding stent rings 44 and allows each stent ring 44 to be partially collapsed after deployment. The secondary constraining members 170 extend through the secondary constraining member lumens 124 (see FIG. 10). In some embodiments, for example as illustrated in FIG. 4B, the secondary constraining members 170 may be divided into two zones allowing independent control of various portions of the longitudinal length of the main body 12. Thus, once a first portion corresponding with a first zone 172 of constraining member of the tubular member 20 is appropriated positioned, it may be released to maintain correct placement while the second zone 174 retains a partially constrained orientation to facilitate placement of the side branches 14. The number of zones defined may include one zone, two zones, or more than two zones.

The secondary constraining members 170 may pass through the tubular member 20 in some embodiments. In order to limit leakage through the tubular member once removed, the secondary constraining members 170 may pass through the tubular member 20 in a column 28 (see FIGS. 2A and 2B). Because the secondary constraining members 170 are supported by the elongate member 110, the elongate member 110 may also be positioned in the column 28. Once the elongate member 110 and secondary constraining members 170 are removed, the column 28 collapses and seals any leakage points that may have been formed by the secondary constraining members 170. The elongate member 110 may include a plurality of exits from the secondary constraining member lumens 124 spaced longitudinally along the elongate member 110 to correspond to the position of each stent ring 44 and constraining member receiver 50. The secondary constraining members 170 are coupled to the delivery handle 200 for facilitating controlled retraction and expansion of the main body 12.

The secondary constraining members 170 may be implemented in order to pull the tubular member 20 away from the vessel wall in order to reposition the main body 12, to allow perfusion through the side branch vessels while the placement and delivery of the implantable device 10 is still being performed, or for visualization of the main body 12 within the vessel (e.g., via fluoroscopy). For example, the main body 12 may have a first diameter corresponding to the delivery configuration and a second diameter corresponding to the deployed configuration, the second diameter being greater than the first configuration. When the secondary constraining members 170 are activated to constrain the main body 12, the main body may include an intermediate diameter that is less than the second diameter and greater than the first diameter and corresponding to the partially constrained configuration.

Referring to FIGS. 5A-6, guide members 220 are illustrated extending through the main body 12 and to the catheter olive 130. The guide members 220 are pre-cannulated through the apertures 32 of the main body 12 prior to insertion into the patient. FIG. 6 illustrates only one aperture 32 of one of the columns 28 being fenestrated with a guide member 220 for simplicity. It is understood that each column 28 includes a corresponding guide member 220 cannulating one of the apertures 32 associated with that column 28. As illustrated in FIG. 6, the guide member 220 is pre-cannulating the aperture closest to the first end 22 of the main body 12. By pre-cannulating the aperture 32 of the column 28, the column 28 is more readily accessible for delivery of the side branch 14 and the cannulation of the remaining apertures 32 within the column 28 is facilitated. The guide members 220 can include various indications for identifying the column 28 with which it is associated. For example, the guide members 220 may be indicated by varying colors, materials, markings, textures, and so forth.

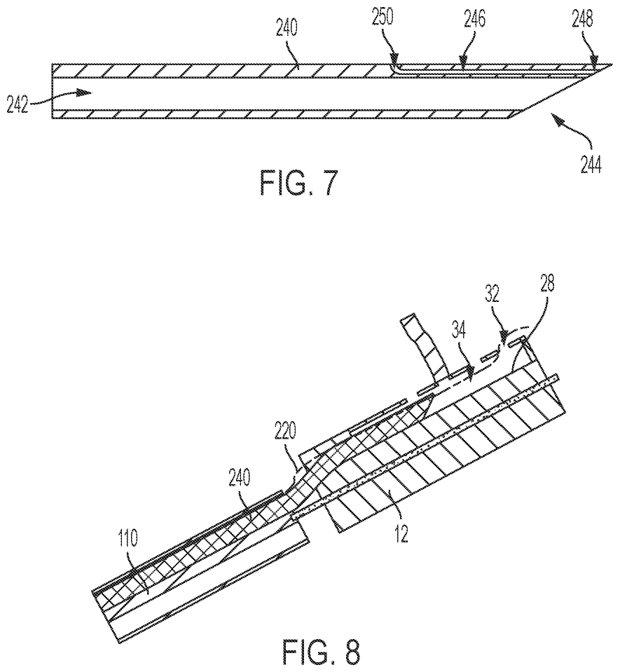

Referring to FIG. 7, a cross section of an exchange catheter 240 is illustrated. The exchange catheter 240 includes a central lumen 242 extending through the exchange catheter 240 with an opening at the first end 244. The first end 244 may include a tapered profile, such as a wedge-shape, in order to facilitate insertion into and/or dilation of a lumen (e.g., the secondary lumen 30 of the column 28). The exchange catheter further defines a peripheral lumen 246. The peripheral lumen 246 extends along the exchange catheter 240 at the first end 244. The peripheral lumen 246 includes a first opening 248 at the first end 244 of the exchange catheter 240 and a second opening 250 spaced from the first end 244 at a position between the ends of the exchange catheter 240. The second opening 250 exits the exchange catheter 240 at an outer surface along the outer circumference of the exchange catheter (i.e., not at the longitudinal ends). The peripheral lumen 246 is operable to house the guide member 220. Thus, the guide member 220 operates to guide the exchange catheter 240 to a desired position.

More specifically, FIG. 8 illustrates the exchange catheter 240 being advanced into the main body 12 from the elongate member 110. In some embodiments, as illustrated in FIG. 8, the exchange catheter 240 exits the elongate member 110 at and end of the elongate member, or in other embodiments, the elongate member 110 includes openings 118 positioned through the side wall of the elongate member 110 exiting from the elongate member 110 at a position between the longitudinal ends of the elongate member 110 (see FIG. 3). The guide member 220 is shown extending into the secondary lumen 30 of the column 28 and through the aperture 32 positioned closest to the first end 22 of the main body 12. The guide member 220 acts as a rail along which the exchange catheter 240 is translated via the peripheral lumen 246. Because the peripheral lumen 246 exits the exchange catheter at the tip of the tapered portion at the first end 244, the exchange catheter 240 is operable to slide into the secondary lumen 30 of the main body 12 and dilate the column 28. The exchange catheter 240 is operable to be advanced to the end of the column at the sealed end as illustrated. The exchange catheter 240 includes central lumen 242 for advancing various other components of the delivery system 100 as is discussed hereafter.

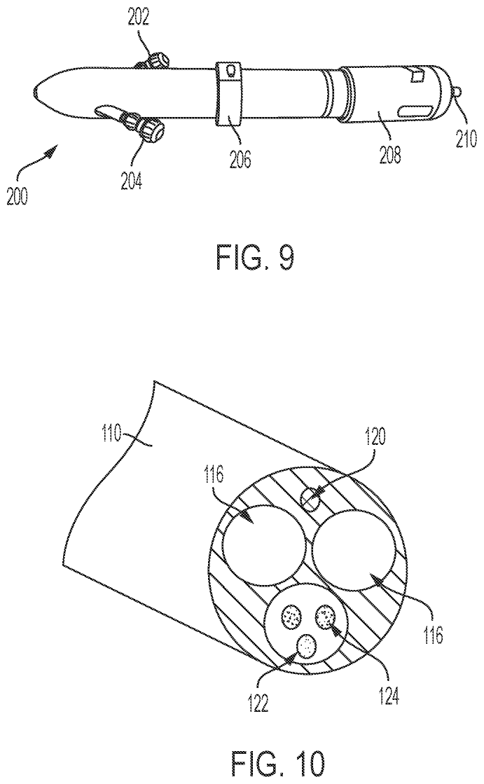

FIG. 9 illustrates a delivery handle 200. The delivery handle 200 includes a first port 202 that hermitically seals a second port 204 that hermitically seals, at least one knob 206 for controlling the constraining wires 170. The first port 202 may include guide members 220 extending therethrough for each column 28 on one lateral side of the main body 12 and the second port 204 may include guide members 220 extending therethrough for each column 28 on another lateral side of the main body 12. The exchange catheter 240 may be loaded onto the guide members 220 and then inserted into the respective port 202, 204. The side branches may also be inserted and advanced to the target site via the respective ports 202, 204. The main body 12 may also be advanced and deployed via the delivery handle 200. For example, the constraining member 150 may be released via a second knob 208. The delivery handle may include a primary access port 210 through which various components of the delivery system 100 may be delivered and manipulated.

FIGS. 11A-11C illustrate various embodiments of the main body 12. FIG. 11A illustrates a main body 12 that can be deployed at least partially within in a second implantable device 300. The second implantable device 300 may include a branched device, for example, for deployment in the iliac arteries. FIG. 11B illustrates a main body 12 with a tapered profile at the second end 24 of the main body. A second implantable device 300 may be deployed within a portion of the main body 12, for example, at the tapered portion. FIG. 11C illustrates a main body 12 including a branched portion at the second end 24. The branched portion of the second end 24 may be implemented with second devices 300 for implantation in branched anatomy, for example, into the iliac arteries. The delivery sequence of the various implantable device 10, 300 may occur in either direction, for example sequentially from the top downward or from the bottom upward in the abdominal aorta. It is understood that the entire delivery method can be performed, for implantation in the abdominal aorta, through the femoral artery. However, various components and other may be delivered from a second entry point.

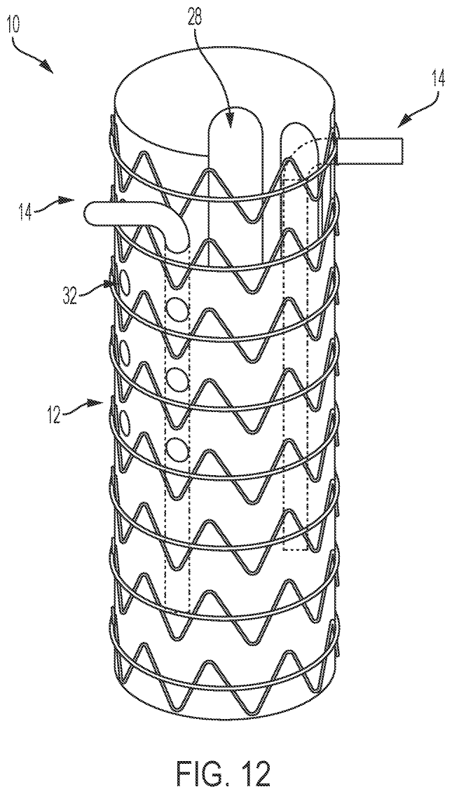

FIG. 12 is an illustration of the main body 12 with two side branches 14 deployed through the apertures 32 and extend through the columns 28 and out the column opening 34 (see FIG. 2B). The length of the side branch 14 can be selected such that the side branch extends a predetermined distance into the side branch vessel and such that the side branches 14 extend out through the column opening 34. By having the side branch opening extending out through the column opening 34, the column 28 is maintained patent and does not seal at the column opening, thus allowing perfusion through the side branches 14. In some embodiments, the main body 12 may include an internal stent (not shown) coupled to the tubular member 20 within the primary lumen 26. The internal stent can be bonded to the columns 28. The internal stent may be constrained to a compressed configuration, which is operable to maintain the columns 28 in a patent configuration. Once the internal stent is released and expanded, the columns 28 are collapsed and sealed such that fluids cannot flow therethrough. In those columns with the side branch 14 positioned therein, the side branch maintains patency of the column when the internal stent is released.

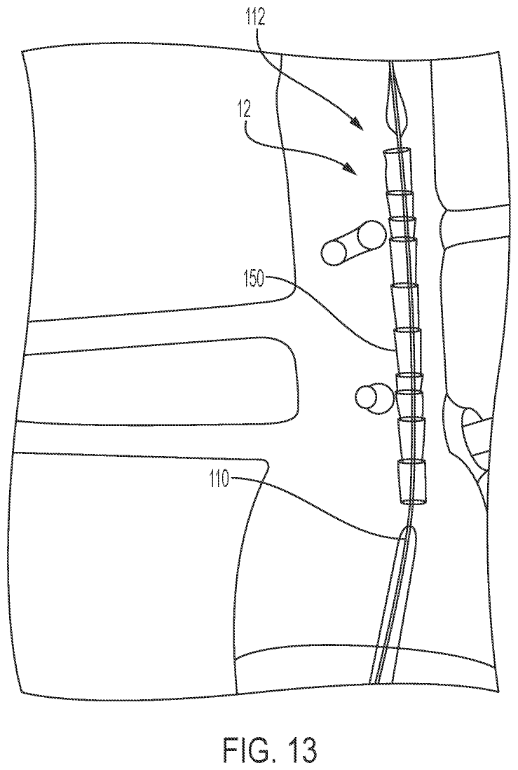

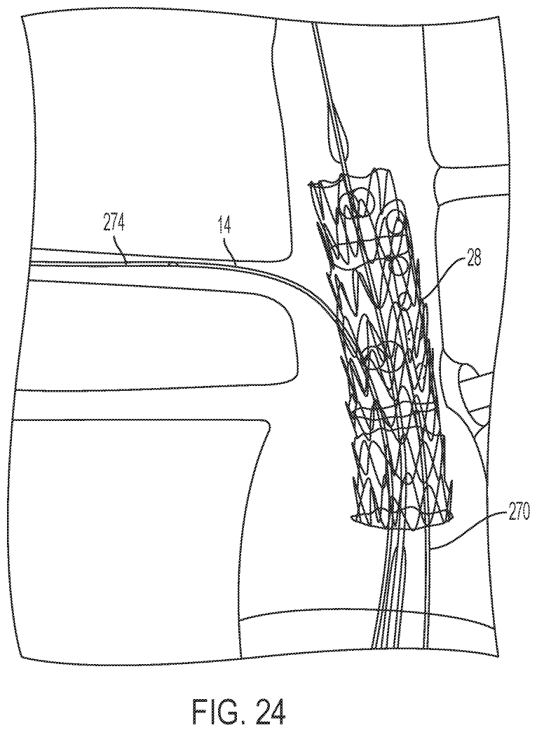

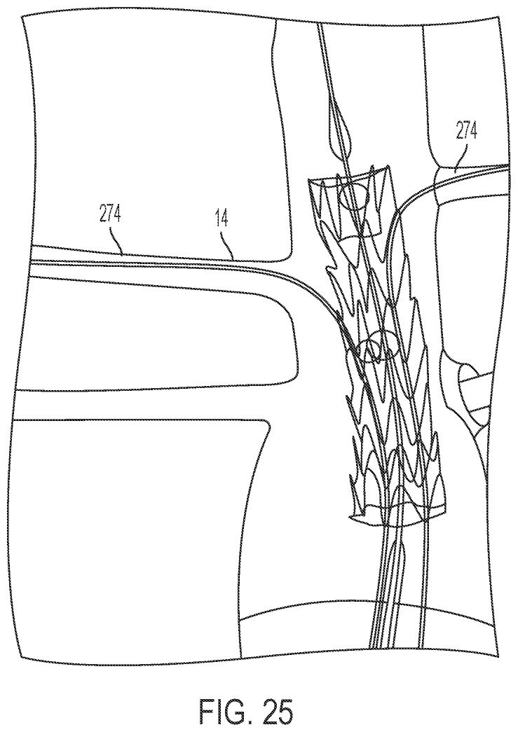

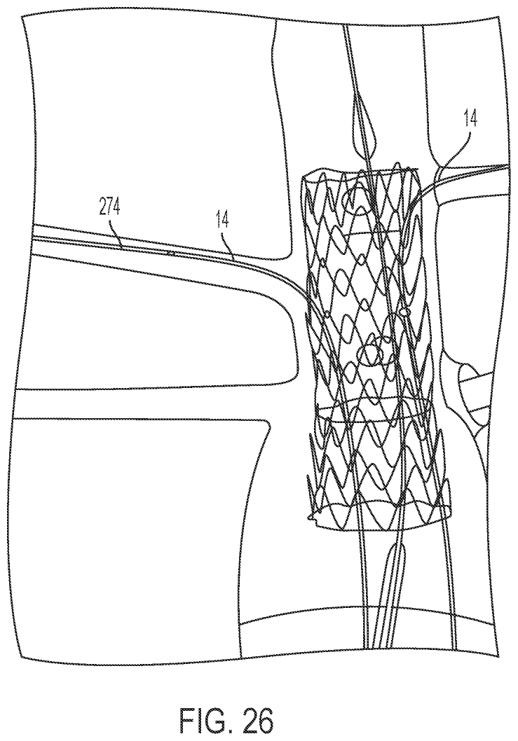

Referring now to FIGS. 13-27, an example method of delivery of an implantable device is illustrated. Although FIGS. 13-27 illustrate and are discussed with reference to an example of an implantable device being delivered and deployed at an example abdominal aorta, it is understood that the method is limited to the abdominal aorta. It is noted that specific features are shown in more detail in some FIGS. in order to highlight the specific function at a specific step of the procedure while others may be hidden so as to simplify the illustrations for understanding. However, a combination of various features may be present at a specific step even if it is not explicitly illustrated in a figure. The methods shown and described herein can be performed from a single access site.

FIG. 13 illustrates a main body 12 of an implantable device 10 being advanced to the target site (e.g., the abdominal aorta). The main body 12 is positioned about an elongate member 110 at the first end 112. The main body 12 is constrained to the delivery configuration via a constraining member 150.

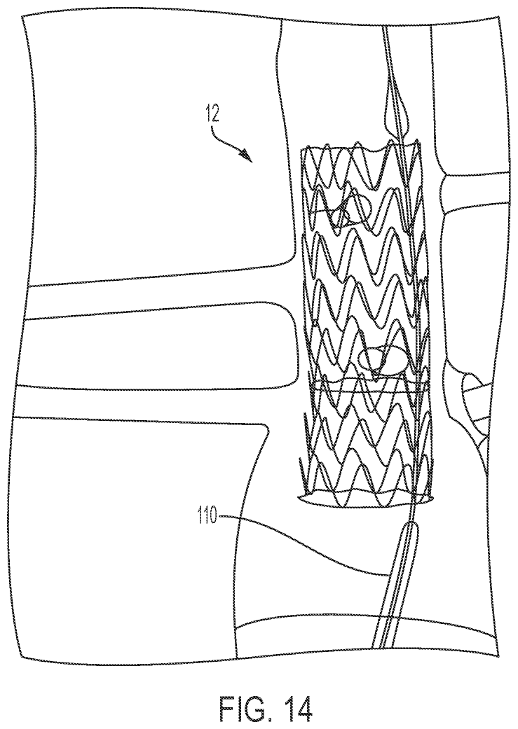

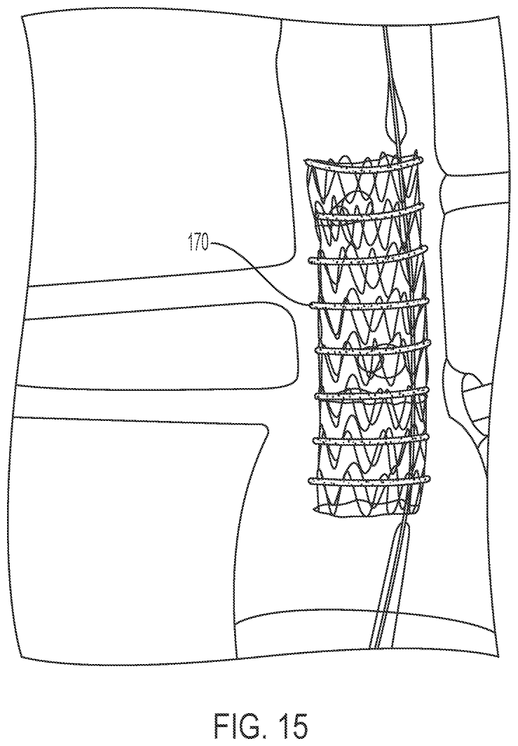

FIG. 14 illustrates the main body 12 being released from the constraining member 150 and expanded to an expanded configuration. FIG. 15 illustrates the secondary constraining members 170 being activated to partially to partially constrain the main body 12. This allows the positioning of the main body 12 to be fine-tuned at the target site.

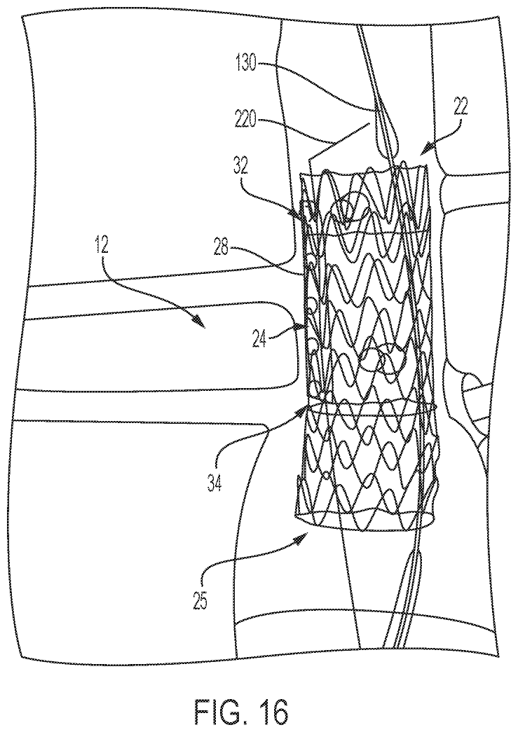

FIG. 16 illustrates main body 12 including a column 28 with apertures 32 longitudinally spaced along the main body 12 providing access to the secondary lumen 30 of the column 28. A guide member 220 extends into the second opening 25 of the main body 12, into the column opening 34, through the column 28, out an aperture 32 closest to the first end 22 of the main body 12, and extending to the catheter olive 130 at which the guide member 220 is retained.

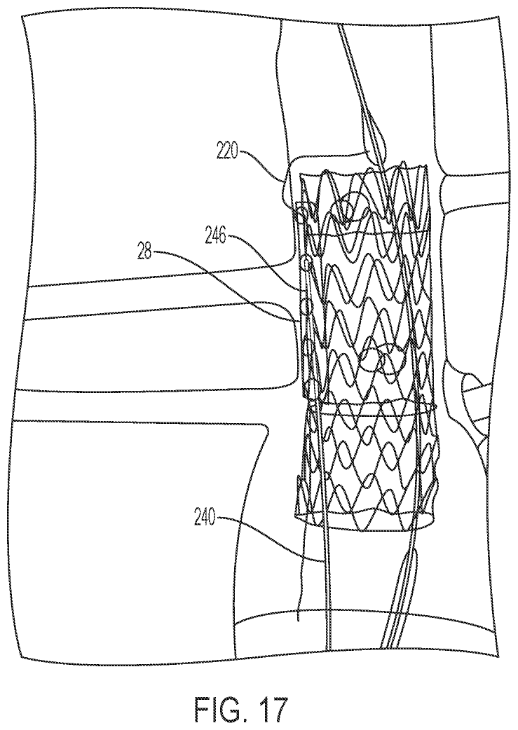

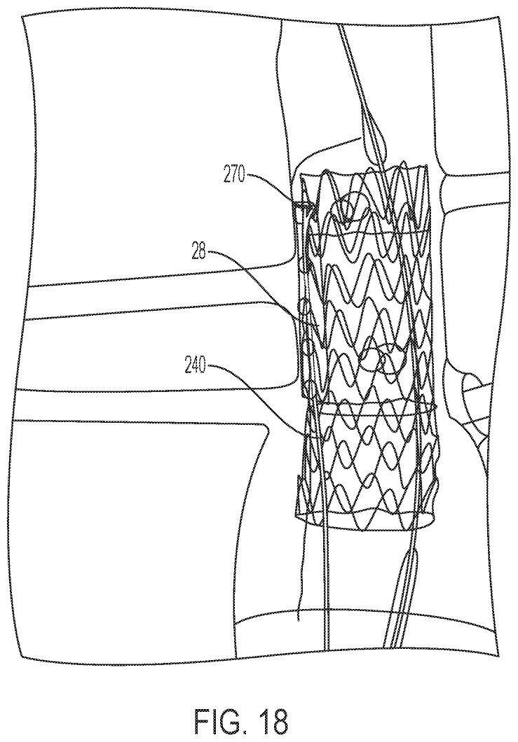

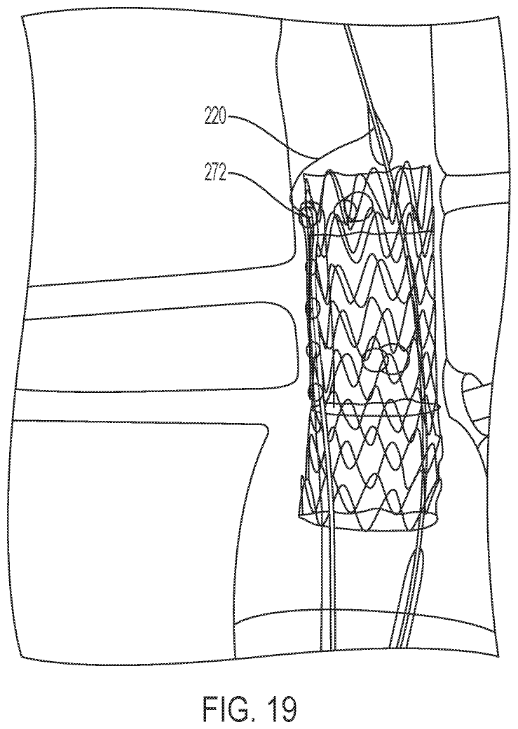

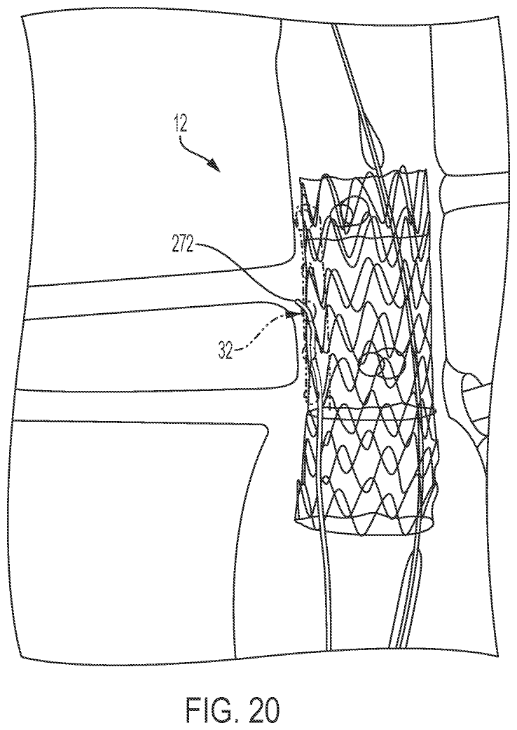

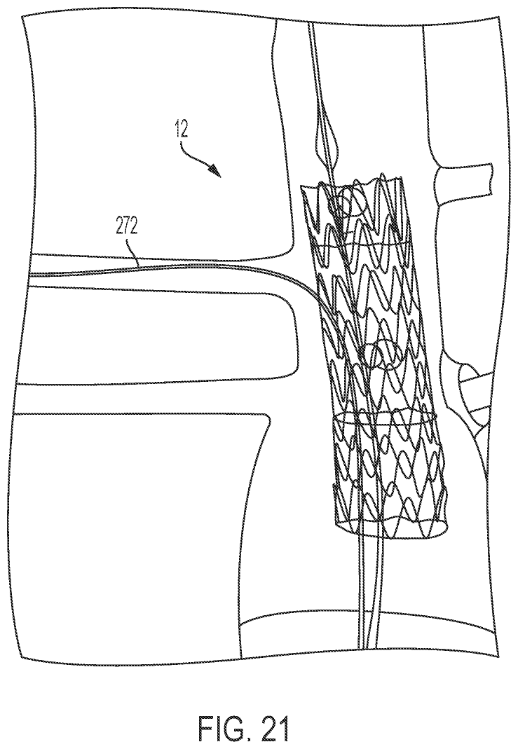

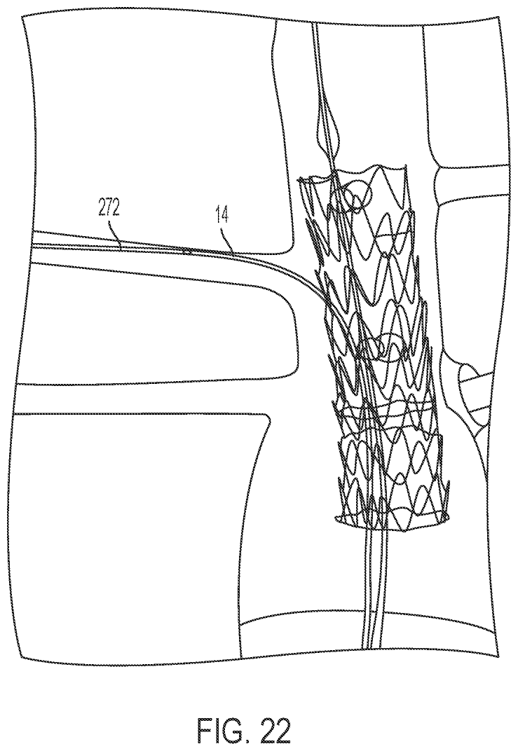

FIG. 17 illustrates the guide member 220 acting as a guide or rail for the exchange catheter 240 to be advanced into the column 28. As illustrated the guide member 220 is positioned in the peripheral lumen 246 of the exchange catheter 240 (see FIGS. 7 and 8). FIG. 18 illustrates the exchange catheter 240 being positioned at the sealed end of the column 28 and a first guidewire 270 being advanced through the central lumen 242 (See FIG. 7) of the exchange catheter 240. The first guidewire 270 may be a compliant guidewire with a curved tip. Referring to FIG. 19, the exchange catheter 240 is then removed. In some embodiments, a curved catheter 272 may be inserted over the first guidewire 270. The curved catheter 272 and the first guidewire 270 are not engaged with the guide member 220. The curved catheter 272 and the first guidewire 270 are oriented such that the curved tips are curving toward a radially outward position. FIG. 20 illustrates the curved catheter 272 and first guidewire 270 being pulled axially away from the first end 22 of the main body 12. The physician selects the appropriate aperture 32 for accessing the side branch vessel. The curved catheter 272 will travel over the apertures 32 when being retracted axially, However, when advanced forward toward the first end 22 of the main body 12, the curved catheter 272 and first guidewire 270 will engage the aperture 32. Once the aperture 32 is engaged, further advancing of the curved catheter 272 will advance the curved catheter 272 through the aperture 32 (see FIG. 21). The curved catheter 272 is then advanced into the side branch vessel. Once the curved catheter 272 is appropriately placed, the first guidewire 270 may be retracted and a second guidewire 274 may be advanced. The second guidewire 274 may be stiffer relative to the first guidewire 270. The curved catheter 272 is then removed while the second guidewire 274 is retained in the side branch lumen (see FIG. 22) through the aperture 32. Although the guide member 220 is not shown in all of these illustrations, it is understood that the guide member 220 may be retained throughout the method so as to regain entry or repeat steps as necessary.

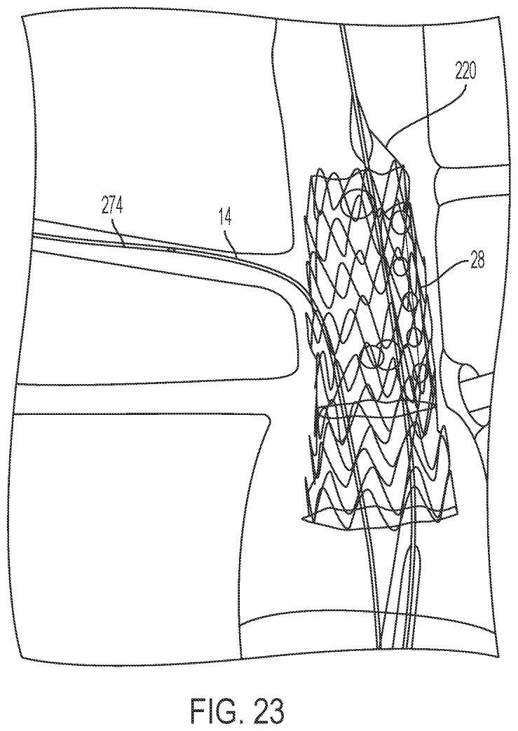

With further reference to FIG. 22, the side branch 14 is advanced along the second guidewire 274 to the target site. FIGS. 23 through 26 provide a similar procedure for placement of a second side branch 14 at the second side branch vessel.

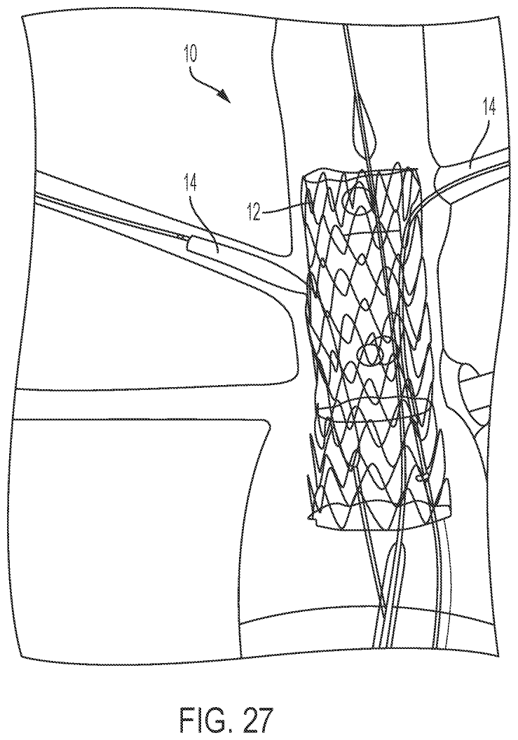

FIG. 27 illustrates one of the side branches 14 deployed with the main body 12. Once the side branches 14 are in position, either individually or each side branch 14, the side branches can be deployed. The side branches 14 can be released from a constraining member and self-expand or can be expanded (e.g., balloon expansion). Once the side branches 14 are deployed, the main body 12 may be finally released (e.g., releasing of the secondary constraining member 170 and the delivery system 100 and its components may be removed.

It is understood that the procedure can be implemented in a de novo procedure or as a reintervention. The method is largely the same for both procedures. As previously discussed, the implantable device 10 may be implemented with various other implantable components for engaging other anatomy such as branched device and so forth (as seen in FIGS. 11A-11C).

A variety of material sets may be implemented for the graft members, including known vascular graft and stent graft materials. Polymers, biodegradable and natural materials can be used for specific applications. And, a variety of manufacturing techniques may be implemented to form the graft members, including extruding, coating, wrapping, combinations thereof, and others.

A biocompatible material for the graft components, discussed herein, may be used. In certain instances, the graft may include a fluoropolymer, such as a polytetrafluoroethylene (PTFE) polymer or an expanded polytetrafluoroethylene (ePTFE) polymer. In some instances, the graft may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

Examples of suitable synthetic polymers include, but are not limited to nylon, polyacrylamide, polycarbonate, polyformaldehyde, polymethylmethacrylate, polytetrafluoroethylene, polytrifluorochlorethylene, polyvinylchloride, polyurethane, elastomeric organosilicon polymers, polyethylene, polypropylene, polyurethane, polyglycolic acid, polyesters, polyamides, their mixtures, blends and copolymers are suitable as a graft member. In one embodiment, the graft is made from a class of polyesters such as polyethylene terephthalate including DACRON® and MYLAR® and polyaramids such as KEVLAR®, polyfluorocarbons such as polytetrafluoroethylene (PTFE) with and without copolymerized hexafluoropropylene (TEFLON® or GORE-TEX®), and porous or nonporous polyurethanes. In another embodiment, the graft comprises expanded fluorocarbon polymers (especially PTFE) materials. Included in the class of preferred fluoropolymers are polytetrafluoroethylene (PTFE), fluorinated ethylene propylene (FEP), copolymers of tetrafluoroethylene (TFE) and perfluoro (propyl vinyl ether) (PFA), homopolymers of polychlorotrifluoroethylene (PCTFE), and its copolymers with TFE, ethylenechlorotrifluoroethylene (ECTFE), copolymers of ethylene-tetrafluoroethylene (ETFE), polyvinylidene fluoride (PVDF), and polyvinylfluoride (PVF). Especially preferred, because of its widespread use in vascular prostheses, is ePTFE. In another embodiment, the graft comprises a combination of the materials listed above. In another embodiment, the graft is substantially impermeable to bodily fluids. The substantially impermeable graft can be made from materials that are substantially impermeable to bodily fluids or can be constructed from permeable materials treated or manufactured to be substantially impermeable to bodily fluids (e.g. by layering different types of materials described above or known in the art). In one embodiment, the main body and branch members, as described above, are made from any combinations of the materials above. In another embodiment, the main body and branch members, as described above, comprise ePTFE.

The stents, as described, may be provided in the form of a series of rings arranged generally coaxially along the graft body. In some embodiments, as described, may be generally cylindrical when restrained and/or when unrestrained and comprise helically arranged undulations having plurality of helical turns. The undulations preferably are aligned so that they are “in-phase” with each other. More specifically, undulations comprise apices in opposing first and second directions. When the undulations are in-phase, apices in adjacent helical turns are aligned so that apices can be displaced into respective apices of a corresponding undulation in an adjacent helical turn. In one embodiment, the undulations have a sinusoidal shape. In another embodiment, the undulations are U-shaped. In another embodiment, the undulations are V-shaped. In another embodiment, the undulations are ovaloid shaped.

In various embodiments, the stent can be fabricated from a variety of biocompatible materials including commonly known materials (or combinations of materials) used in the manufacture of implantable medical devices. Typical materials include 316L stainless steel, cobalt-chromium-nickel-molybdenumiron alloy (“cobalt-chromium”), other cobalt alloys such as L605, tantalum, nitinol, or other biocompatible metals. In one embodiment, any stent-graft described herein is a balloon expandable stent-graft. In another embodiment, any stent-graft described herein is a self-expanding stent-graft. In another embodiment, the stent is a wire wound stent. In another embodiment, the wire wound stent includes undulations, or a repeating, undulating pattern of apices.

The wire wound stent can be constructed from a reasonably high strength material, e.g., one which is resistant to plastic deformation when stressed. In one embodiment, the stent member comprises a wire which is helically wound around a mandrel having pins arranged thereon so that the helical turns and undulations can be formed simultaneously, as described below. Other constructions also may be used. For example, an appropriate shape may be formed from a flat stock and wound into a cylinder or a length of tubing formed into an appropriate shape or laser cutting a sheet of material. In another embodiment, said stent is made from a super-elastic alloy. There are a variety of disclosures in which super-elastic alloys such as nitinol are used in stents.

A variety of materials variously metallic, super elastic alloys, such as Nitinol, are suitable for use in these stents. Primary requirements of the materials are that they be suitably springy even when fashioned into very thin sheets or small diameter wires. Various stainless steels which have been physically, chemically, and otherwise treated to produce high springiness are suitable as are other metal alloys such as cobalt chrome alloys (e.g., ELGILOY®), platinum/tungsten alloys, and especially the nickel-titanium alloys generically known as “nitinol”.

Nitinol is especially preferred because of its “super-elastic” or “pseudo-elastic” shape recovery properties, i.e., the ability to withstand a significant amount of bending and flexing and yet return to its original form without permanent deformation. These metals are characterized by their ability to be transformed from an austenitic crystal structure to a stress-induced martensitic structure at certain temperatures, and to return elastically to the austenitic shape when the stress is released. These alternating crystalline structures provide the alloy with its super-elastic properties.

Other suitable stent materials include certain polymeric materials, particularly engineering plastics such as thermotropic liquid crystal polymers (“LCP's”). These polymers are high molecular weight materials which can exist in a so-called “liquid crystalline state” where the material has some of the properties of a liquid (in that it can flow) but retains the long range molecular order of a crystal. The term “thermotropic” refers to the class of LCP's which are formed by temperature adjustment. LCP's may be prepared from monomers such as p,p′-dihydroxy-polynuclear-aromatics or dicarboxy-polynuclear-aromatics. The LCP's are easily formed and retain the necessary interpolymer attraction at room temperature to act as high strength plastic artifacts as are needed as a foldable stent. They are particularly suitable when augmented or filled with fibers such as those of the metals or alloys discussed below. It is to be noted that the fibers need not be linear but may have some preforming such as corrugations which add to the physical torsion enhancing abilities of the composite.

Any of a variety of bio-active agents may be implemented with any of the foregoing. For example, any one or more of (including portions thereof) the device 10 may comprise a bio-active agent. Bio-active agents can be coated onto one or more of the foregoing features for controlled release of the agents once the device 10 is implanted. Such bio-active agents can include, but are not limited to, thrombogenic agents such as, but not limited to, heparin. Bio-active agents can also include, but are not limited to agents such as anti-proliferative/antimitotic agents including natural products such as vinca alkaloids (e.g., vinblastine, vincristine, and vinorelbine), paclitaxel, epidipodophyllotoxins (e.g., etoposide and teniposide), antibiotics (e.g., dactinomycin (actinomycin D), daunorubicin, doxorubicin, and idarubicin), anthracyclines, mitoxantrone, bleomycins, plicamycin (mithramycin) and mitomycin, enzymes (e.g., L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); anti-proliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine {cladribine}); platinum coordination complexes (e.g., cisplatin and carboplatin), procarbazine, hydroxyurea, mitotane, aminoglutethimide; hormones (e.g., estrogen); anti-coagulants (e.g., heparin, synthetic heparin salts and other inhibitors of thrombin); anti-platelet agents (e.g., aspirin, clopidogrel, prasugrel, and ticagrelor); vasodilators (e.g., heparin, aspirin); fibrinolytic agents (e.g., plasminogen activator, streptokinase, and urokinase), aspirin, dipyridamole, ticlopidine, clopidogrel, abciximab; antimigratory agents; antisecretory agents (e.g., breveldin); anti-inflammatory agents, such as adrenocortical steroids (e.g., cortisol, cortisone, fludrocortisone, prednisone, prednisolone, 6α-methylprednisolone, triamcinolone, betamethasone, and dexamethasone), non-steroidal agents (e.g., salicylic acid derivatives, such as aspirin); para-aminophenol derivatives (e.g., acetaminophen); indole and indene acetic acids (e.g., indomethacin, sulindac, and etodalac), heteroaryl acetic acids (e.g., tolmetin, diclofenac, and ketorolac), arylpropionic acids (e.g., ibuprofen and derivatives), anthranilic acids (e.g., mefenamic acid and meclofenamic acid), enolic acids (e.g., piroxicam, tenoxicam, phenylbutazone, and oxyphenthatrazone), nabumetone, gold compounds (e.g., auranofin, aurothioglucose, and gold sodium thiomalate); immunosuppressives (e.g., cyclosporine, tacrolimus (FK-506), sirolimus (rapamycin), azathioprine, and mycophenolate mofetil); angiogenic agents (e.g., vascular endothelial growth factor (VEGF)), fibroblast growth factor (FGF); angiotensin receptor blockers; nitric oxide donors; anti-sense oligonucleotides and combinations thereof; cell cycle inhibitors, m TOR inhibitors, growth factor receptor signal transduction kinase inhibitors; retinoids; cyclin/CDK inhibitors; HMG co-enzyme reductase inhibitors (statins); and protease inhibitors.

Numerous characteristics and advantages of the present invention have been set forth in the preceding description, including preferred and alternate embodiments together with details of the structure and function of the invention. The disclosure is intended as illustrative only and as such is not intended to be exhaustive. It will be evident to those skilled in the art that various modifications may be made, especially in matters of structure, materials, elements, components, shape, size and arrangement of parts within the principals of the invention, to the full extent indicated by the broad, general meaning of the terms in which the appended claims are expressed. To the extent that these various modifications do not depart from the spirit and scope of the appended claims, they are intended to be encompassed therein. In addition to being directed to the embodiments described above and claimed below, the present invention is further directed to embodiments having different combinations of the features described above and claimed below.

It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An implantable device comprising:
a tubular member having a first end and a second end, the tubular member forming a primary lumen having a first opening at the first end of the tubular member and a second opening at the second end of the tubular member, the tubular member including a column positioned within the primary lumen and forming a secondary lumen, the tubular member defining a plurality of apertures opening into the secondary lumen at positions longitudinally between the first end and the second end of the tubular member, the column having a column opening; and
a stent member supporting the tubular member.

2. The implantable device of claim 1, wherein the tubular member includes a first graft member defining the primary lumen and a second graft member coupled to the first graft member forming the column and defining the secondary lumen between the first and second graft members.

3. The implantable device of claim 2, wherein the secondary lumen opens into the primary lumen at the proximal opening of the secondary lumen.

4. The implantable device of claim 1, wherein the secondary lumen is collapsible.

5. The implantable device of claim 1, wherein the secondary lumen is unsupported by a stent.

6. The implantable device of claim 1, wherein the stent member includes a plurality of stent rings spaced longitudinally along the tubular member.

7. The implantable device of claim 6, wherein each of the plurality of apertures is separated from each other by at least one of the plurality of stent rings.

8. The implantable device of claim 1, wherein each aperture of the plurality of apertures includes a circular shape or a shape defining a rounded portion and a substantially flat portion.

9. The implantable device of claim 1, wherein the tubular member includes a plurality of secondary lumens extending longitudinally along at least a portion of the tubular member and circumferentially spaced from each other.

10. The implantable device of claim 1, further comprising a constraining member receiver positioned surrounding at least a portion of the stent member.

11. The implantable device of claim 1, wherein the tubular member includes a scallop at the first end.

12. The implantable device of claim 1, wherein the secondary lumen extends along a secondary lumen axis that extends longitudinally at an angle greater than zero relative to an axis of the primary lumen.

13. An implantable device comprising:
a tubular member having a first end and a second end, the tubular member forming a primary lumen having a first opening at the first end of the tubular member and a second opening at the second end of the tubular member, the tubular member including a plurality of columns each defining a secondary lumen, each column of the plurality of columns being circumferentially spaced from each other, each secondary lumen having an aperture defined through the tubular member at a position between the first end and the second end and having a column opening proximate the second end of the tubular member, each column being operable to collapse under hydrostatic pressure; and
a stent member supporting the tubular member.

14. A delivery system comprising:
an implantable device including a main body and a side branch, the main body including a tubular member and a stent member supporting the tubular member, the tubular member forming a primary lumen having a first opening and a second opening, the tubular member forming a secondary lumen having an aperture defined through the tubular member and having a proximal opening proximal the second opening of the tubular member;
an elongate member having a first end and a second end, the main body of the implantable device being positioned at the first end of the elongate member, the elongate member defining a delivery lumen extending along at least a portion of a longitudinal length of the elongate member and through which the side branch is operable to be delivered;
a catheter olive positioned at the first end of the elongate member such that the main body of the implantable device is positioned longitudinally between a leading tip of the catheter olive and the second end of the elongate member;
a constraining member positioned about the main body and constraining the main body to a delivery configuration; and
a secondary constraining member positioned about the stent of the main body, the secondary constraining member operable to constrain the main body to a partially constrained configuration.

15. The delivery system of claim 14, further comprising a guide member extending from the delivery lumen of the elongate member, through the secondary lumen of the tubular member from the proximal opening and through the aperture, and coupled to the catheter olive.

16. The delivery system of claim 15, further comprising an exchange catheter coupled to the guide member and operable to be advance along the guide member.

17. The delivery system of claim 16, further comprising:
a first guidewire operable to be delivered through the exchange catheter;
a curved catheter operable to be advanced over the first guidewire; and
a second guidewire that is stiffer than the first guidewire, the second guidewire operable to be advanced through the curved catheter, the side branch operable to be advanced to a target site along the second guidewire.

18. A delivery system comprising:
an implantable device including a main body and a side branch, the main body including tubular member and stent member supporting the tubular member, the tubular member forming a primary lumen having a first opening and a second opening, the tubular member including a column defining a secondary lumen and having an aperture defined through the tubular member and having a column opening proximal the second opening of the tubular member;
an elongate member having a first end and a second end, the main body of the implantable device being positioned proximal to the first end of the elongate member, the elongate member defining a lumen through which the side branch is operable to be delivered;
a catheter olive positioned at the first end of the elongate member such that the main body of the implantable device is positioned longitudinally between a leading tip of the catheter olive and the second end of the elongate member;
a constraining member positioned about the main body and constraining the main body to a delivery configuration; and
a guide member extending from the lumen of the elongate member, through the secondary lumen of the tubular member from the proximal opening and through the aperture, and coupled to the catheter olive.

19. The delivery system of claim 18, further comprising a secondary constraining member positioned about the stent of the main body, the secondary constraining member operable to constrain the main body to a partially constrained configuration.

20. The delivery system of claim 19, wherein the stent member of the main body includes a plurality of stent rings, wherein the secondary constraining member includes a plurality of wires, wherein a wire of the plurality of wires corresponds to a corresponding stent ring of the plurality of stent rings.

21. (canceled)
